(19) United States
(12) Patent Application Publication
(10) Pub. No.: US 2026/0199188 A1
(43) Pub. Date: Jul. 16, 2026

(54) CONNECTOR WITH HERMETIC CLOSURE, WHICH CAN BE OPENED BY PRESSURE AND IS AUTOMATICALLY RECLOSABLE WITH IMPROVED MANOEUVRABILITY

(71) Applicant: PAOLO GOBBI FRATTINI s.r.l., Milano (IT)

(72) Inventor: Paolo Giuseppe GOBBI FRATTINI, Sondalo (SO) (IT)

(73) Assignee: PAOLO GOBBI FRATTINI s.r.l., Milano (IT)

(21) Appl. No.: 19/447,220

(22) Filed: Jan. 13, 2026

(30) Foreign Application Priority Data
Jan. 15, 2025 (IT) 202025000000162

(51) Int. Cl. A61J 1/20; A61J 1/14

(52) U.S. Cl. CPC A61J 1/2055; A61J 1/1406; A61J 1/1481; A61J 1/2044

(57) ABSTRACT

A connector (100) with hermetic closure openable and automatically reclosable for devices intended for collecting and dispensing fluids for pharmaceutical and/or nutritional use comprises a main body (1) provided with a longitudinal axial bore (5, 6). The main body (1) has an end (7) provided with a transversal wall (8) for closing said axial bore (5,6), said end being crossed by a thin axial slit (9). A thin membrane (10), that can be broken by external axial pressure and is automatically reclosable when said axial pressure ceases, keeps said axial slit (9) normally closed. The connector (100) is provided with an external collar (11) with a terminal piece (12) with external thread (13), on which a luer of a syringe or other device can be threaded, said external collar (11) being provided with at least one radial fin (14) protruding outside. (FIG. 1).

The present invention relates to a connector with hermetic closure, which can be opened by pressure and is automatically reclosable for devices for collecting and dispensing pharmaceutical and/or nutritional fluid substances.

From EP 2 667 839B1, there are known hermetic connectors that can be opened by pressure and are automatically reclosable, which connectors are used for filling and emptying flexible bags for therapeutic pharmaceutical and/or nutritional fluid substances, as well as for the mutual connection of other devices intended for similar aims.

In particular, still from EP 2 667 839B1, there are known connectors of the aforesaid type, wherein a filling and dispensing end is provided with a closing plug made of elastic material, normally hermetically closed, which can be perforated by axial pressure via a terminal luer of a needleless syringe (or similar device) screwed on a threaded terminal piece of said connector end.

Sometimes, due to the small dimensions of such connectors, the screwing of the syringe luer on the connector end is complicated and uncomfortable, with ensuing inconvenience.

Aim of the present invention is improving the existing connectors for such use, so making the screwing of the luer to the connector simpler, more convenient and faster.

According to the present invention such aim has been achieved by adding one or more outside protruding fins to said connector end, in a non-threaded back piece thereof, according to what is claimed in claim 1.

In this way, the mutual screwing operation between the connector and the syringe luer or other device becomes possible, easy and comfortable, both manually and through a suitable tool or apparatus.

The features of the present invention will be made more evident by the following detailed description of an embodiment of the present invention shown by way of non-limiting example in the appended drawings, wherein:

FIG. 1 shows a connector according to the invention, in a perspective view;

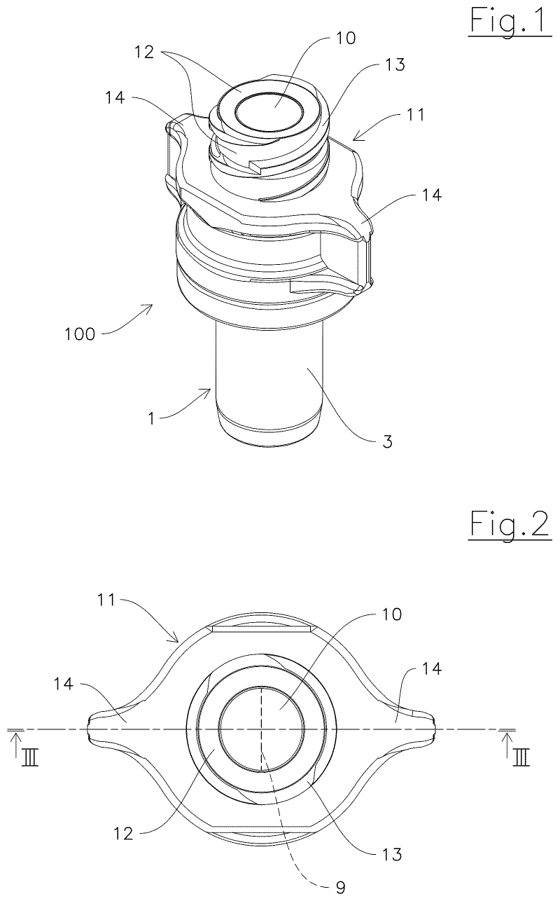

FIG. 2 shows the same connector, seen in top view with respect to FIG. 1;

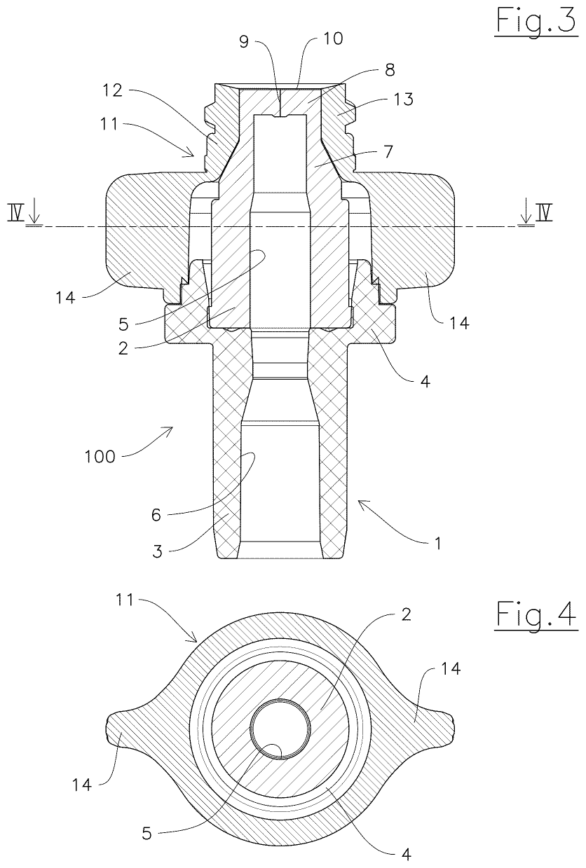

FIG. 3 shows the same connector, in an axial longitudinal section view taken along line III-III in FIG. 1;

FIG. 4 shows the connector in a transversal section taken along line IV-IV of FIG. 3.

The connector shown in the drawings, wherein it is overall indicated with numeral 100, comprises a main body 1 made of axially consecutive tubular upper and lower pieces 2 and 3 (according to what is shown in FIGS. 1 and 3), wherein the two pieces are inserted into a chalice top 4 of the second, and are provided with respective axial aligned and communicating bores 5 and 6.

The upper piece 2 is made of elastically deformable transparent plastic material, having a top end 7 converging toward a transversal wall 8 which forms an upper closing plug of the bore 5, and is crossed by a thin axial slit 9. In a unique body with the upper piece 2 a thin membrane 10 is realized in, which can be broken by an external axial pressure, and is automatically reclosable when such axial pressure ceases, which membrane 10 keeps said axial slit 9 normally closed.

On the other hand, the lower piece 3 is made of substantially rigid transparent plastic material.

The two tubular pieces 2 and 3 are kept together by an external collar 11 made of rigid transparent plastic material, which is screwed around the chalice top 4 of the tubular piece 3, and in this way tightened around the upper end 7 of the tubular piece 2. The collar 11 is provided with a terminal 12 with external thread 13, on which a luer of a syringe or other device can be screwed.

All the features described up to now can also be derived from EP 2667839 B1, EP 3636241 B1 and EP 4000588 B1.

Innovatively, in the connector according to the invention, the external collar 11 is provided with a pair of radial fins 14 (or even just one fin or more than two fins), protruding outside from opposed sides, intended for easing the screwing of a luer of a syringe or other device on the threaded terminal portion 12 of the connector 100 when the connector 100 is used for introducing and/or withdrawing a fluid substance into or from a flexible bag applied to the lower tubular piece 3 of the connector 100.

1. Connector (100) with hermetic closure for devices intended for collecting and dispensing fluids for pharmaceutical and/or nutritional use, comprising a main body (1) provided with a longitudinal axial bore (5, 6), wherein said main body (1) has an end (7) provided with a transversal wall (8) for closing said axial bore (5, 6), said end being crossed by a thin axial slit (9), and wherein there is provided a thin membrane (10), that can be broken through external axial pressure and is automatically reclosable when said axial pressure ceases, said membrane (10) keeping said axial slit (9) normally closed, wherein there is provided an external collar (11) provided with a terminal piece (12) with external thread (13), on which a luer of a syringe or other device can be screwed, characterized in that said external collar (11) is provided with at least one radial fin (14) protruding outside.

2. Connector according to claim 1, characterized in that said at least one radial fin (14) consists of a pair of fins (14) protruding from opposed sides of said collar (11).

3. Connector according to claim 1, characterized in that said main body (1) is formed by two axially consecutive tubular pieces (2, 3) which are inserted one (2) into a chalice portion (4) of the second (3) and provided with axial aligned and communicating bores (5, 6).

4. Connector according to claim 3, characterized in that said tubular piece (2) is made of elastically deformable transparent plastic material.

5. Connector according to claim 4, characterized in that said tubular pieces (2, 3) are kept together by said external collar (11), and said external collar (11) is screwed around the chalice portion (4) of the tubular piece (3) and tightened around said upper end (7) of the tubular piece (2), and wherein said collar (11) is provided with a terminal portion (12) with external thread (13) on which a syringe luer can be screwed.
